(19) United States
(12) Patent Application Publication
(10) Pub. No.: US 2021/0104442 A1
(43) Pub. Date: Apr. 8, 2021

(54) STABLE MIXED OXIDE CATALYSTS FOR DIRECT CONVERSION OF ETHANOL TO ISOBUTENE AND PROCESS FOR MAKING

(71) Applicants: Archer Daniels Midland Company, Decatur, IL (US); Washington State University, Pullman, WA (US)

(72) Inventors: Junming Sun, Pullman, WA (US); Changjun Liu, Pullman, WA (US); Yong Wang, Pullman, WA (US); Kevin Martin, Mt. Zion, IL (US); Padmesh Venkitasubramanian, Forsyth, IL (US); Colin Smith, Pullman, WA (US)

(21) Appl. No.: 17/124,207

(22) Filed: Dec. 16, 2020

Related U.S. Application Data
(62) Division of application No. 15/860,815, filed on Jan. 3, 2018.
Application No. 15/860,815 is a continuation of application No. 14/683,787, filed on Apr. 10, 2015, now abandoned.

(51) Int. Cl. H01L 21/8238; B01J 37/08; B01J 21/06; B01J 21/04; B01J 23/06; B01J 27/02; C07C 1/20; H01L 21/265; H01L 27/092; H01L 29/66

(52) U.S. Cl. CPC H01L 21/823842; H01L 29/66545; B01J 21/06; B01J 21/04; B01J 23/06; B01J 27/02; C07C 1/20; H01L 21/26513; H01L 21/823821; H01L 21/82385; H01L 27/0922; H01L 27/0924; H01L 29/66537; C07C 2523/06; B01J 37/08

(57) ABSTRACT

ZnₓZr_{y}O_{z} mixed oxide catalysts having improved stability for the conversion of ethanol to isobutene are described, together with methods for making such catalysts.

### CROSS-REFERENCE TO RELATED APPLICATIONS

The present application is a divisional of U.S. application Ser. No. 15/860,815, filed Jan. 3, 2018, as a continuation of U.S. application Ser. No. 14/683,787, filed Apr. 10, 2015, which in turn was a national stage entry of International Application No. PCT/US2013/062784, filed Oct. 1, 2013, now published as WO 2014/070354, which directly claimed the benefit of U.S. Provisional Patent Application Ser. No. 61/720,433 filed Oct. 31, 2012.

### TECHNICAL FIELD

The present invention relates generally to renewable process alternatives for the production of isobutene. More particularly, the present invention relates to processes for the direct conversion of ethanol to isobutene and to the catalysts used therein, and still more particularly relates to the methods used for making such catalysts.

### BACKGROUND ART

As background, biomass is considered as a CO₂ neutral energy carrier, and is one of the most abundant and renewable of natural resources. In recent years, both as a result of market conditions as well as in response to a variety of governmental initiatives and mandates, biomass transformation to produce biofuels has attracted significant effort and investment, with the result that bioethanol has become a commodity biofuel product. With the increased availability and reduced cost of bioethanol, opportunities have been explored to use bioethanol not just as a biofuel, but as a feedstock for making a variety of renewable source-derived chemicals.

The most commercially advanced initiative has been the dehydration of bioethanol to produce ethylene, though the conversions of bioethanol to propylene and isobutene have also been studied. Propylene is an important industrial intermediate for the production of propylene oxide and polypropylene, for example, while isobutene is similarly widely used for the production of a variety of industrially important products, such as butyl rubber for example. Both of propylene and isobutene are obtained through the catalytic or steam cracking of fossil feedstocks, and the development of a commercially viable process for the direct conversion of bioethanol to either material would accordingly be of great interest as fossil resources are depleted and/or become more costly to use—especially in consideration of increased demand for both of propylene and isobutene.

Efforts to convert ethanol to propylene have largely centered on the use of zeolite or modified zeolite catalysts, with isobutene being produced largely as a secondary consideration as a co-product. A very recent example of work on a non-zeolitic, modified metal oxide catalyst is found in Mizuno et al., “One—path and Selective Conversion of Ethanol to Propene on Scandium-modified Indium Oxide Catalysts”, Chem. Lett., vol. 41, pp. 892-894 (2012), wherein a proposed direct pathway involving ethanol dehydrogenation to acetaldehyde, followed by acetone formation through condensation and decomposition and the subsequent hydrogenation of acetone to form isopropanol, then the eventual dehydration of isopropanol to form propylene was investigated.

Mizuno et al. found that In203-based oxides were active for the direct conversion of ethanol to propylene, but that needed improvements in the stability of the catalyst could be achieved by the addition of certain modifying metals to prevent the reduction of the indium oxides to indium metal and through the presence of water vapor to reduce coke formation. Mizuno additionally observed that a cofeed of hydrogen increased the propylene yield in relation to other co-products such as isobutene, through promotion of the selective hydrogenation-dehydration of acetone to propylene. While percentage yields of isobutene on a carbon basis were observed of up to 24.0 percent on an unmodified indium oxide catalyst and up to just less than 23 percent on a modified indium catalyst, the modified catalysts were again preferred because considerably higher propylene yields were observed in some instances despite lower isobutene yields.

Previously, we have reported a hard-template method to synthesize ZnₓZr_{y}O_{z} mixed oxides for the direct and high yield conversion of ethanol (from the fermentation of carbohydrates from renewable source materials, including biomass) to isobutene, wherein we added ZnO to ZrO₂ to selectively passivate zirconia's strong Lewis acidic sites and weaken Brönsted acidic sites while simultaneously introducing basicity. Our objectives were to suppress ethanol dehydration and acetone polymerization, while enabling a surface basic site-catalyzed ethanol dehydrogenation to acetaldehyde, an acetaldehyde to acetone conversion via aldol-condensation/dehydrogenation, and a Brönsted and Lewis acidic/basic site-catalyzed acetone-to-isobutene reaction pathway.

High isobutene yields were in fact realized, but unfortunately, as later experienced by Mizuno et al. in their efforts to produce propylene from ethanol, we found that further improvements in the catalyst's stability were needed. Unlike Mizuno et al., however, we have determined that these improvements could be realized without adding modifying metals and without a reduction in the initial high activity (100 percent ethanol conversion) we had observed in these mixed oxide catalysts.

### SUMMARY OF THE INVENTION

The following presents a simplified summary of the invention in order to provide a basic understanding of some of its aspects. This summary is not an extensive overview of the invention and is intended neither to identify key or critical elements of the invention nor to delineate its scope. The sole purpose of this summary is to present some concepts of the invention in a simplified form as a prelude to the more detailed description that is presented later.

With this in mind, the present invention in one aspect concerns a ZnₓZr_{y}O_{z} mixed oxide catalyst having improved stability for the conversion of ethanol to isobutene, exhibiting less than 10 percent loss in isobutene selectivity over a period of 200 hours on stream.

In another embodiment, the catalysts of the present invention exhibit less than 5 percent loss in isobutene selectivity over the same time on stream.

In still another embodiment, the catalysts of the present invention exhibit less than 2 percent loss in isobutene selectivity over the same time on stream.

In another aspect, the present invention concerns a process for making a ZnₓZr_{y}O_{z} mixed oxide catalyst having improved stability for the conversion of ethanol to isobutene, comprising forming a solution of one or more Zn compounds, combining one or more zirconium-containing solids with the solution of one or more Zn compounds, drying the wetted solids, then calcining the dried solids.

### BRIEF DESCRIPTION OF THE DRAWING

FIG. 1 depicts a proposed reaction pathway for the conversion of ethanol to isobutene using a catalyst of the present invention.

### DESCRIPTION OF EMBODIMENTS

In a first aspect, a ZnₓZr_{y}O_{z} mixed oxide catalyst is provided having improved stability for the conversion of ethanol to isobutene, exhibiting less than 10 percent loss in isobutene selectivity over a period of 200 hours on stream under atmospheric pressure (less than 35 kPa (5 psig)) and at 450° C., while the ethanol conversion was kept at 100%. Preferably, however, the catalyst exhibits less than 5 percent loss in isobutene selectivity over a period of 200 hours on stream, and more preferably less than 2 percent.

Our previous ZnₓZr_{y}O_{z} mixed oxide catalysts were highly active, demonstrating high selectivity (greater than 80 percent to isobutene) with substantially full conversion of ethanol. After further testing over a number of hours on-stream, however, it was found that isobutene selectivity dropped more quickly than desired, for example by more than 10 percent over a period of thirty hours on-stream.

When we made a ZnₓZr_{y}O_{z} mixed oxide catalyst for comparison by an alternate method, though, using the same values for x, y and z as before, it was surprisingly determined that the catalyst made by a different method exhibited much greater stability—showing, for example, less than 2 percent loss in isobutene selectivity over a period of up to 200 hours on stream. Thermogravimetric analysis confirmed that much less coke was formed on catalysts made by the new method as compared to the old, though the improvement in stability was not realized at the expense of activity, as the catalysts made by the former and the new methods gave substantially the same initial high activity that had previously been reported.

Our previous method was a “hard template” or “confined space synthesis” method generally of the character used by Jacobsen et al., “Mesoporous Zeolite Single Crystals”, Journal of the American Chemical Society, vol. 122, pp. 7116-7117 (2000), wherein nanozeolites were prepared.

More particularly, the same carbon black (BP 2000, Cabot Corp.) was used as a hard template for the synthesis of nanosized ZnₓZr_{y}O_{z} mixed oxides, rather than nanozeolites as in Jacobsen et al. Prior to use, the BP 2000 template was dried at 180° C. overnight. Calculated amounts of zirconyl nitrate hydrate (Sigma-Aldrich, greater than 99.8% purity) and Zn(NO₃)₂.6H₂O (Sigma-Aldrich, greater than 99.8% purity) were dissolved in a given amount of water and sonicated for 15 minutes to produce a clear solution with desired concentrations of Zn and Zr. About 25 grams of the obtained solution were then mixed with 6.0 grams of the preheated BP 2000 to achieve incipient wetness, and the mixture was transferred to a ceramic crucible and calcined at 400 degrees Celsius for 4 hours, followed by ramping the temperature to 550 degrees Celsius (at a ramp rate of 3 degrees Celsius/minute) and holding at 550 degrees Celsius for another 20 hours. Nanosized white powders were obtained, having a mean particle size of less than 10 nanometers.

The catalysts made by such former method are further described in Sun et al., “Direct Conversion of Bio-ethanol to Isobutene on Nanosized ZnₓZr_{y}O_{z} Mixed Oxides with Balanced Acid-Base Sites”, Journal of the American Chemical Society, vol. 133, pp 11096-11099 (2011), along with findings related to the character of the mixed oxide catalysts formed thereby and the performance of the catalysts given certain Zn/Zr ratios, residence times and reaction temperatures.

While the present invention concerns a different method for making ZnₓZr_{y}O_{z} mixed oxide catalysts of a generally very similar character, it is expected that the mixed oxide catalysts made by the inventive method and further characterized below can be run under comparable conditions as reported in this publication with at least the same or better outcomes.

Briefly, the ZnₓZr_{y}O_{z} mixed oxide catalysts of the present invention will be characterized by a Zn/Zr ratio (x:y) of from 1:100 to 10:1, preferably from 1:30 to 1:1, especially 1:20 to 1:5, and still more preferably 1:12 to 1:10.

Parenthetically, in the present application where any range of values is given for any aspect or feature of the catalysts of the present invention or any process described for using the catalysts of the present invention, the given ranges will be understood as disclosing and describing all subranges of values included within the broader range. Thus, for example, the range of 1:100 to 10:1 will be understood as disclosing and describing not only the specific preferred and more preferred subranges given above, but also every other subrange including a value for x between 1 and 10 and every other subrange including a value for y between 1 and 100.

The catalysts made by the new method are consistent in their particle size with the catalysts described in the journal article, namely, comprising aggregates of less than 10 nm-sized particles with a highly crystalline structure. The Zn oxide component is again highly dispersed on the Zr oxide component.

Some characteristic differences have, however, been observed between catalysts of equivalent Zn/Zr ratios made by the inventive and prior methods. For example, average crystallite size as calculated based on the Scherer equation will typically be larger, for example, 8.4 nanometers for a Zn₁Zr₁₀O₂ mixed oxide catalyst prepared according to the present method as compared to 4.8 nanometers for a Zn₁Zr₁₀O₂ mixed oxide catalyst prepared according to the former method.

The same Zn₁Zr₁₀O₂ mixed oxide catalyst prepared according to the present method also has a smaller surface area, approximately 49 square meters per gram, as compared to approximately 138 square meters per gram for a Zn₁Zr₁₀O₂ mixed oxide catalyst prepared according to the former method.

One further, compositional difference was also observed between catalysts prepared by the two methods, in that the ZnₓZr_{y}O_{z} mixed oxide catalysts of the present invention preferably are substantially sulfur-free, containing less than 0.14 weight percent of sulfur, as compared to, for example, 3.68 weight percent of sulfur in the same Zn₁Zr₁₀O₂ mixed oxide catalyst prepared according to the former method.

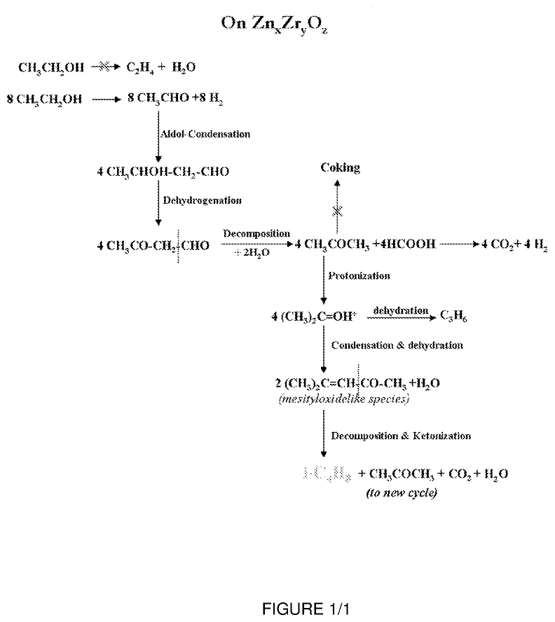

The ZnₓZr_{y}O_{z} mixed oxide catalysts of the present invention have improved stability for the conversion of ethanol to isobutene, for which a proposed (but not limiting) reaction pathway is illustrated in FIG. 1; while the contributions if any of the larger crystallite size and smaller surface area to this improved stability are not presently understood, it is nevertheless believed that at least the much reduced sulfur content of the inventive catalysts does contribute materially to this improved stability.

In this regard, to further explore the much-reduced coking and improved stability behaviors exhibited by the inventive catalysts, infrared analyses of adsorbed pyridine were performed on a Zn₁Zr₁₀O₂ mixed oxide catalyst prepared according to the former method and on a comparable Zn₁Zr₁₀O₂ mixed oxide catalyst prepared according to the present invention. The infrared spectra revealed that at 250 degrees Celsius, both Lewis and Brönsted acidic sites were significantly lower for the inventive catalyst as compared to the mixed oxide catalyst made by the former method. At a desorption temperature of 350 degrees Celsius, almost no Brönsted acidic sites were observed on the inventive catalyst and the number of stronger Lewis acidic sites was also lower. The presence of sulfur in the former catalysts—presumably left behind from the Cabot BP 2000 furnace black hard template after the template's being substantially removed by a controlled combustion as taught by Jacobsen et al—thus appeared to have contributed to the presence of a number of stronger Lewis and Brönsted acidic sites on catalysts made by the former method and in turn to a greater degree of acidic site-catalyzed coking of catalysts made according to the former method.

Accordingly, while in one aspect the invention concerns ZnₓZr_{y}O_{z} mixed oxide catalysts having improved stability for the conversion of ethanol to isobutene, exhibiting less than 10 percent loss in isobutene selectivity over a period of 200 hours on stream, from a different, compositional perspective the invention particularly concerns ZnₓZr_{y}O_{z} mixed oxide catalysts containing less than 0.14 percent by weight of sulfur. Preferably, still more stable catalysts are provided, having a sulfur content of less than 0.01 percent by weight, and still more preferably the catalysts will have a sulfur content of less than 0.001 percent by weight.

Catalysts as described may be made by a process broadly comprising, in certain embodiments, forming a solution of one or more Zn compounds, combining one or more zirconium-containing solids with the solution of one or more Zn compounds so that the solution wets the zirconium-containing solids to a state of incipient wetness, drying the wetted solids, then calcining the dried solids. In other embodiments, a solution is formed of one or more Zr compounds, the solution is combined with one or more Zn-containing solids so that the solution wets the Zn-containing solids to a state of incipient wetness, the wetted solids are dried and then the dried solids are calcined. In principle, provided the zinc and zirconium compounds and solids in these embodiments do not contain sulfur, any combination of zinc and zirconium materials and any solvent can be used that will permit the zinc and zirconium components to mix homogeneously whereby, through incipient wetness impregnation, one of the zinc or zirconium components are well dispersed on a solid of the other component for subsequent drying and conversion to the oxide forms through calcining.

The conditions and times for the drying and calcining steps will depend, of course, on the particular zinc and zirconium materials and solvent used, but in general terms, the drying step can be accomplished in a temperature range of from 60 degrees Celsius to 200 degrees Celsius over at least 3 hours, while the calcining can take place at a temperature of from 300 degrees Celsius to 1500 degrees Celsius, but more preferably a temperature of from 400 to 600 degrees Celsius is used. The calcination time can be from 10 minutes to 48 hours, with from 2 to 10 hours being preferred.

In still other embodiments, catalysts as described herein can be prepared by a hard template method as described in our prior publication, except that a suitable very low sulfur content carbon is used for the hard template such that the finished catalyst will contain not more than 2 percent by weight of sulfur, especially not more than 0.5 percent by weight of sulfur and still more preferably will contain not more than 0.1 weight percent (by total weight of the catalyst) of sulfur. A variety of such very low sulfur carbons are available commercially from various suppliers; in general, the lower the sulfur content, the better for forming the highly active, stable mixed oxide catalysts of the present invention.

Processes for converting ethanol to isobutene using the inventive catalysts may be conducted in a manner and under conditions described in our prior publication, or in a manner and under conditions described in Mizuno et al or the several other prior publications concerned with the production of products inclusive of isobutene from ethanol. In this regard, while Mizuno et al. is particularly directed to the production of propylene from ethanol, it is nevertheless considered to be well within the capabilities of those skilled in the art to determine what conditions embraced by Mizuno et al. or other similar references will be most appropriate to produce isobutene among the possible products, without undue experimentation. Accordingly, a detailed description of process details for using the more stable mixed oxide catalysts of the present invention need not be undertaken herein. Nevertheless, as an example of an ethanol to isobutene process using the inventive catalysts, a continuous fixed bed reactor or flow bed reactor can be used. The reaction temperature may be in a range from 350 to 700 degrees Celsius, preferably, in a range from 400 to 500 degrees Celsius, and the WHSV can be in a range from 0.01 hr⁻¹ to 10 hr⁻¹, preferably from 0.05 hr⁻¹ to 2 hr⁻¹. Ethanol/water solution with steam to carbon ratios from 0 to 20, preferably from 2 to 5 can be used.

The present invention is further illustrated by the following non-limiting examples:

### Example 1 and Comparative Example 1

Commercial zirconium hydroxide was dried at 120 degrees Celsius for more than 5 hours. Calculated amounts of Zn(NO₃)₂ (from Sigma-Aldrich, more than 99.8 percent purity) were dissolved in water to form a series of clear solutions. Dried zirconium hydroxide (also from Sigma-Aldrich, more than 99.8 percent purity) was then mixed with the solutions in turn by incipient wetness, in order to form wet powders impregnated with Zn in certain proportions to the zirconium in the form of the dried zirconium hydroxide powder. The wetted powders were then dried at 80 degrees Celsius for 4 hours, followed by calcination at 400 degrees Celsius for 2 hours and at 600 degrees Celsius for 3 hours to obtain a series of ZnₓZr_{y}O_{z} catalysts by the new method.

For comparison, a mixed oxide catalyst was prepared using the comparatively higher sulfur Cabot BP-2000 carbon as a hard template. The BP-2000 carbon template was first dried at 180 degrees Celsius overnight, and the amounts of zirconyl nitrate hydrate and Zn(NO₃)₂.6H₂O needed to form a Zn₁Zr₈O₂ catalyst were dissolved in a given amount of water and sonicated for 15 minutes to produce a clear solution. About 25 grams of the solution were mixed with 6.0 grams of preheated, dried BP-2000 carbon to achieve incipient wetness, and the mixture was transferred to a ceramic crucible, heated to 400 degrees Celsius at 3 degrees Celsius per minute and then held at 400 degrees Celsius for 4 hours. The temperature was then increased at 3 degrees Celsius to 550 degrees Celsius, and the final calcination accomplished by holding the catalyst at 550 degrees for a further 20 hours.

Ethanol to isobutene runs were conducted with the catalysts thus prepared in a fixed-bed stainless steel reactor, having an inside diameter of 5 millimeters. A given amount of catalyst was packed between quartz wool beds. A thermocouple was placed in the middle of the catalyst bed to monitor the reaction temperatures. Before beginning the reaction, the catalyst beds were first pretreated by flowing 50 ml/minute of nitrogen at 450 degrees Celsius through the catalyst over a half hour, then a mixture of ethanol/water at steam to carbon ratios from 1 to 5 was introduced into an evaporator at 180 degrees Celsius by means of a syringe pump and carried into the reactor by the flowing nitrogen carrier gas. Meanwhile, the product line was heated to in excess of 150 degrees Celsius before a cold trap, to avoid condensing the liquid products in the product line.

A Shimadzu 2400 gas chromatograph equipped with an auto sampling valve, HP-Plot Q column (30 m, 0.53 mm, 40 μm) and flame ionization detector was connected to the line between the reactor outlet and cold trap to collect and analyze the products in the effluent gas. After the cold trap, an online micro-GC (MicroGC 3000A equipped with molecular sieves 5A, plot U columns and thermal conductivity detectors) was used to analyze the product gases specifically, using nitrogen as a reference gas.

An ethanol/water solution (steam to carbon ratio of 2.5) was then supplied by flowing N₂ to the reactor at a weight hourly space velocity (WHSV) of 0.95 hr⁻¹. The ethanol concentration was 15.1 percent by weight, and the reaction temperature was 450 degrees Celsius. Ethanol conversion was 100% for both the inventive and prior hard template catalysts throughout the time on stream for each, but isobutene selectivity dropped for the hard template catalyst from about 53 percent to about 40 percent after 27 hours on stream while acetone selectivity increased, other products remaining consistent. In contrast, after 200 hours on stream, isobutene selectivity for the inventive catalyst declined by less than 2 percent.

Thermogravimetric and differential scanning calorimetry analysis of the recovered, spent catalysts showed a weight loss of 5.6 weight percent and an exothermal peak at 380 degrees Celsius for the hard template catalyst after only 30 hours on stream, indicating combustion of the coke that had deposited on the catalyst. By comparison, only about 0.7 weight percent of coke was detected on the inventive catalyst after 207 hours onstream.

### Examples 2 Through 30

Based on the improved catalyst stability demonstrated in Example 1, a number of additional catalysts were prepared by first drying commercial zirconium hydroxide at 120 degrees Celsius for more than 5 hours. Calculated amounts of Zn(NO₃)₂ (from Sigma-Aldrich, more than 99.8 percent purity) were dissolved in water to form a series of clear solutions. The dried zirconium hydroxide (also from Sigma-Aldrich, more than 99.8 percent purity) was then mixed with the solutions in turn by incipient wetness, in order to form wet powders impregnated with Zn in certain proportions to the zirconium in the form of the dried zirconium hydroxide powder. The wetted powders were then dried at 80 degrees Celsius for 4 hours, followed by calcination at the temperature indicated in Table 1 below for 3 hours, to obtain a series of ZnₓZr_{y}O_{z} catalysts by the new method. Particular reaction conditions, whether the reaction temperature, WHSV or steam to carbon ratio, for example, were then varied to compare the effect on the selectivities to acetone and isobutene at full conversion of the ethanol. For several of the catalysts, some amount of sulfur was purposely doped into the catalyst to assess the effect of sulfur at those certain levels on the selectivities to acetone and to isobutene. Thus, the catalyst for example 27 was doped with 10 ppm of sulfur, while for example 28 the catalyst was doped with 50 ppm of sulfur and for example 29 with 200 ppm (by weight).

TABLE 1
Ethanol to Isobutene Runs

| | | Calcination | Reaction | WHSV | Steam to | | Acetone | Isobutene |
|---|---|---|---|---|---|---|---|---|
| | Zn/Zr | temp | temp | (gₑₜₕₐₙₒₗ/ | carbon | Ethanol | selectivity | selectivity |
| Ex # | ratios | (° C.) | (° C.) | g_{catal}/hr) | ratio | (gas wt %) | (mol %) | (mol %) |
| 2 | 1/6.5 | 550 | 450 | 0.19 | 5 | 1.0 | 3.5 | 46.4 |
| 3 | 1/6.5 | 550 | 425 | 0.08 | 5 | 1.0 | 4.0 | 49.8 |
| 4 | 1/8 | 550 | 450 | 0.19 | 5 | 1.0 | 3.4 | 47.3 |
| 5 | 1/8 | 550 | 415 | 0.08 | 5 | 1.0 | 8.5 | 51.4 |
| 6 | 1/10 | 550 | 450 | 0.19 | 5 | 1.0 | 2.9 | 49.2 |
| 7 | 1/10 | 550 | 425 | 0.08 | 5 | 1.0 | 3.8 | 51.5 |
| 8 | 1/12 | 550 | 450 | 0.19 | 5 | 1.0 | 2.5 | 48.9 |
| 9 | 1/12 | 550 | 450 | 0.08 | 5 | 1.0 | 0.5 | 45.5 |
| 10 | 1/12 | 550 | 425 | 0.08 | 5 | 1.0 | 3.8 | 51.6 |
| 11 | 1/12 | 550 | 415 | 0.08 | 5 | 1.0 | 6.2 | 51.3 |
| 12 | 1/14 | 550 | 450 | 0.19 | 5 | 1.0 | 4.9 | 46.8 |
| 13 | 1/10 | 500 | 450 | 0.19 | 5 | 1.0 | 0.7 | 47.6 |
| 14 | 1/10 | 500 | 475 | 0.19 | 5 | 1.0 | 0 | 41.9 |
| 15 | 1/10 | 500 | 450 | 0.08 | 5 | 1.0 | 0 | 42.7 |
| 16 | 1/10 | 500 | 425 | 0.08 | 5 | 1.0 | 1.2 | 49.3 |
| 17 | 1/10 | 600 | 475 | 0.19 | 5 | 1.0 | 7.2 | 42.3 |
| 18 | 1/10 | 600 | 450 | 0.19 | 5 | 1.0 | 13.7 | 42.1 |
| 19 | 1/10 | 600 | 450 | 0.08 | 5 | 1.0 | 4.3 | 43.8 |
| 20 | 1/10 | 600 | 425 | 0.08 | 5 | 1.0 | 12.9 | 44.8 |
| 21 | 1/10 | 600 | 400 | 0.08 | 5 | 1.0 | 32.6 | 33.1 |
| 22 | 1/10 | 650 | 450 | 0.19 | 5 | 1.0 | 32.2 | 30.1 |
| 23 | 1/10 | 650 | 450 | 0.08 | 5 | 1.0 | 10.6 | 41.8 |
| 24 | 1/10 | 650 | 425 | 0.19 | 5 | 1.0 | 44.9 | 23.0 |
| 25 | 1/10 | 650 | 425 | 0.08 | 5 | 1.0 | 26.1 | 37.4 |
| 26 | 1/10 | 650 | 415 | 0.08 | 5 | 1.0 | 34.1 | 32.3 |
| 27 | 1/10 | 550 | 415 | 0.08 | 5 | 1.0 | 7.3 | 52.1 |
| 28 | 1/10 | 550 | 415 | 0.08 | 5 | 1.0 | 6.3 | 52.4 |
| 29 | 1/10 | 550 | 415 | 0.08 | 5 | 1.0 | 8.4 | 51.2 |
| 30 | 1/8 | 550 | 450 | 0.31 | 2.5 | 15.0 | 2.8 | 53.5 |

1. A process for converting ethanol to products including isobutene, comprising:
contacting ethanol with a ZnₓZr_{y}O_{z} mixed oxide catalyst containing less than 0.14 percent by weight of sulfur, and wherein x:y is from 1:100 to 10:1 and z is a stoichiometric integer for the mixed oxide catalyst, at a temperature in the range of from about 350 to about 700 degrees Celsius and a WHSV in the range of from about 0.01 hr⁻¹ to 10 hr⁻¹, to produce a product mixture including isobutene; and
recovering isobutene from the product mixture.

2. The process of claim 1, wherein the catalyst contains less than 0.01 percent by weight of sulfur.

3. The process of claim 2, wherein the catalyst contains less than 0.001 percent by weight of sulfur.
